(19) United States
(12) Patent Application Publication
(10) Pub. No.: US 2023/0293537 A1
(43) Pub. Date: Sep. 21, 2023

(54) USE OF ACETAMIDE DERIVATIVE IN PREVENTION AND TREATMENT OF ALZHEIMER'S DISEASE

(71) Applicant: ACADEMY OF MILITARY MEDICAL SCIENCES, BEIJING (CN)

(72) Inventors: Yunfeng Li, BEIJING (CN); Liming Zhang, BEIJING (CN); Rumeng Yao, BEIJING (CN); Rifang Yang, BEIJING (CN); Wei Dai, BEIJING (CN)

(21) Appl. No.: 18/014,875

(22) PCT Filed: Jul. 6, 2021
(86) PCT No.: PCT/CN2021/104702

§ 371 Date: Jan. 6, 2023

(30) Foreign Application Priority Data
Jul. 9, 2020 (CN) 202010658043.4

(51) Int. Cl. A61K 31/5377; A61K 31/437; A61K 31/444; A61P 25/28

(52) U.S. Cl. CPC A61K 31/5377; A61K 31/437; A61K 31/444; A61P 25/28

(57) ABSTRACT

The present invention relates to the use of an acetamide derivative in the prevention and treatment of Alzheimer's disease (AD). In particular, the present invention relates to the use of the compound as represented by formula I, and a solvate or a pharmaceutically acceptable salt thereof in the preparation of a drug for the prevention and/or treatment and/or adjuvant treatment of AD,

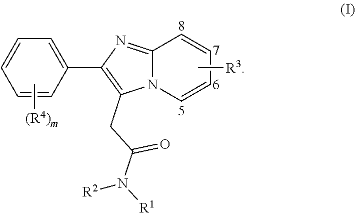

The present application is based on the application with the CN application number of 202010658043.4 and the filing date of Jul. 9, 2020, and claims its priority. The disclosure of this CN application is hereby incorporated into the present application in its entirety.

### TECHNICAL FIELD

The present invention belongs to the field of medicine and chemical industry, and relates to the use of an acetamide derivative in the prevention and treatment of Alzheimer's disease.

### BACKGROUND ART

Alzheimer's disease (AD) is a progressive neurodegenerative disease characterized by insidious onset and impairment of memory and cognitive function. The main clinical manifestations are memory impairment, aphasia, agnosia, impairment of visuospatial skills, executive dysfunction, and personality and behavior changes. According to demographic projections, the number of AD patients worldwide will exceed 100 million by 2050. However, due to the complex pathogenesis of AD, there is currently no effective drug for the treatment of this disease in clinical practice. Therefore, to find safe and efficient anti-AD drugs is an urgent problem to be solved.

### CONTENTS OF THE PRESENT INVENTION

After in-depth research and creative work, the inventors found that TSPO ligands of formula I, especially YL-IPA08 (see Compound 8 in Table 1 for its name and structural formula), have significant anti-AD effects, revealing the important value of the TSPO ligands (e.g., TSPO agonists or TSPO activators) in the prevention and treatment of AD, thereby providing the following invention.

In one aspect, the present application relates to use of a TSPO ligand in the manufacture of a medicament for the prevention and/or treatment and/or adjuvant treatment of AD. In some embodiments, the TSPO ligand is selected from the following compounds of formula I.

In another aspect, the present application relates to use of a compound of formula I, a solvate or pharmaceutically acceptable salt thereof, in the manufacture of a medicament for the prevention and/or treatment and/or adjuvant treatment of AD,

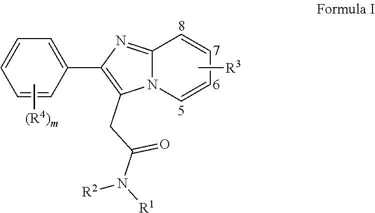

wherein:
R¹ is selected from the group consisting of ethyl, propyl, butyl and methoxyethyl;
R² is selected from the group consisting of benzyl, 2-pyridylmethyl, 3-pyridylmethyl, 4-pyridylmethyl, 2-(4-morpholinyl)ethyl and 3 -(4-morpholinyl)propyl;
R³ is selected from the group consisting of H and C₁₋₆ alkyl;
each R⁴ is independently selected from the group consisting of H, halogen, C₁₋₆ alkyl and C₁-C₆ alkoxy;
m is 0, 1, 2 or 3.

In this context, the pharmaceutically acceptable salt of the compound of formula I may be an acid addition salt or a salt formed with base. The acid addition salt can be an inorganic acid salt, including but not limited to hydrochloride, sulfate, phosphate, or hydrobromide, etc.; or an organic acid salt, including but not limited to acetate, oxalate, citrate, gluconate, succinate, tartrate, p-toluenesulfonate, mesylate, benzoate, lactate, or maleate, etc.; the salt formed with the compound of formula I and base can be an alkali metal salt, including but not limited to lithium salt, sodium salt, or potassium salt, etc.; or an alkaline earth metal salt, including but not limited to calcium salt or magnesium salt; or an organic base salt, including but not limited to diethanolamine salt or choline salt, etc.; or a chiral base salt, including but not limited to alkylphenylamine salt, and the like. In some embodiments, the pharmaceutically acceptable salt of the compound of formula I is a hydrochloride salt thereof.

In some embodiments, in the compound of formula I, R³ is selected from C₁₋₄ alkyl. In some embodiments, the R³ is selected from methyl or ethyl. In some embodiments, R³ is on the 5-, 6-, 7- or 8-position of the imidazo[1,2-a]pyridine ring. In some embodiments, R³ is on the 6- or 7-position of the imidazo[1,2-a]pyridine ring.

In some embodiments, R⁴ in the compound of formula I is selected from the group consisting of fluorine, chlorine, C₁₋₄ alkyl and C₁-C₄ alkoxy. In some embodiments, R⁴ is selected from fluorine, chlorine, methyl, and methoxy, and m is 1 or 2. In some embodiments, R⁴ is chlorine and m is 2. In some embodiments, when R⁴ is chlorine and m is 2, the two chlorines are on the 2- and 3-positions or the 3- and 4-positions of the benzene ring.

In some embodiments, R¹ is ethyl;
R² is 2-pyridylmethyl;
R³ is selected from C₁₋₄ alkyl, and R³ is on the 6- or 7-position of the imidazo[1,2-a]pyridine ring;
each R⁴ is independently selected from the group consisting of fluorine, chlorine, C₁₋₄ alkyl and C₁-C₄alkoxy, m is 2, and the two R⁴ are on the 2- and 3-positions or 3- and 4-positions of the benzene ring.

In some embodiments, R¹ is ethyl;
R² is 2-pyridylmethyl;
R³ is methyl or ethyl, and R³ is on the 6- or 7-position of the imidazo[1,2-a]pyridine ring;
R⁴ is chlorine, m is 2, and the two chlorines are on the 2- and 3-positions or the 3- and 4-positions of the benzene ring.

In some embodiments, the compound of formula I, or the pharmaceutically acceptable salt thereof, is selected from the following compounds, or pharmaceutically acceptable salts thereof:

TABLE 1
Some compounds of the present application or pharmaceutically acceptable salts thereof

| No. | Name | Structural formula |
|---|---|---|
| 1 | N-benzyl-N-ethyl-2-[2-(4-chlorophenyl)-7-methyl- imidazo[1,2-a]pyridin-3-yl]-acetamide | 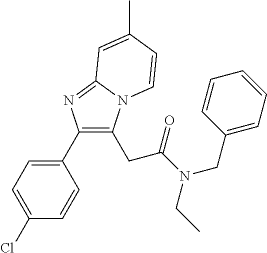 [SMILES: CCN(Cc1ccccc1)C(=O)Cc1c(-c2ccc(Cl)cc2)nc2cc(C)ccn12] |
| 2 | N-ethyl-N-(2-pyridylmethyl)-2-[2-(4- chlorophenyl)-7-methyl-imidazo[1,2-a]pyridin-3- yl]-acetamide•hydrochloride | 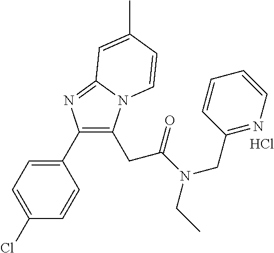 [SMILES: CCN(Cc1ccccn1)C(=O)Cc1c(-c2ccc(Cl)cc2)nc2cc(C)ccn12 ; Cl] |
| 3 | N-ethyl-N-(3-pyridylmethyl)-2-[2-(4- chlorophenyl)-7-methyl-imidazo[1,2-a]pyridin-3- yl]-acetamide•hydrochloride | 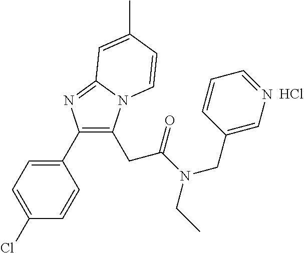 [SMILES: CCN(Cc1cccnc1)C(=O)Cc1c(-c2ccc(Cl)cc2)nc2cc(C)ccn12 ; Cl] |
| 4 | N-ethyl-N-(4-pyridylmethyl)-2-[2-(4- chlorophenyl)-7-methyl-imidazo[1,2-a]pyridin-3- yl]-acetamide·hydrochloride | 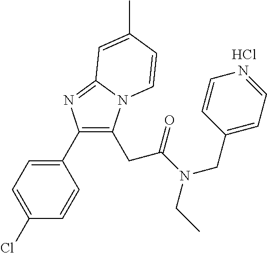 [SMILES: CCN(Cc1ccncc1)C(=O)Cc1c(-c2ccc(Cl)cc2)nc2cc(C)ccn12 ; Cl] |
| 5 | N-(2-methoxyethyl)-N-(2-pyridylmethyl)-2-[2-(4- chlorophenyl)-7-methyl-imidazo[1,2-a]pyridin-3- yl]-acetamide•hydrochloride | 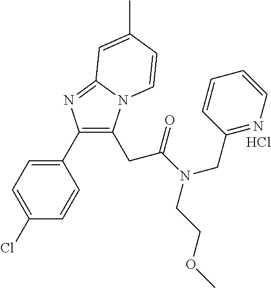 [SMILES: COCCN(Cc1ccccn1)C(=O)Cc1c(-c2ccc(Cl)cc2)nc2cc(C)ccn12 ; Cl] |
| 6 | N-(2-methoxyethyl)-N-(3-pyridylmethyl)-2-[2-(4- methylphenyl)-7-methyl-imidazo[1,2-a]pyridin-3- yl]-acetamide•hydrochloride | 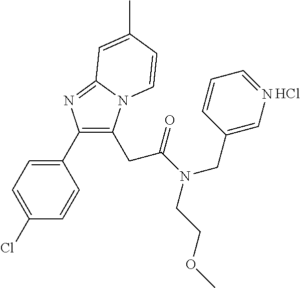 [SMILES: COCCN(Cc1cccnc1)C(=O)Cc1c(-c2ccc(Cl)cc2)nc2cc(C)ccn12 ; Cl] |
| 7 | N-benzyl-N-ethyl-2-[2-(3,4-dichlorophenyl)-7- methyl-imidazo[1,2-a]pyridin-3-yl]-acetamide | 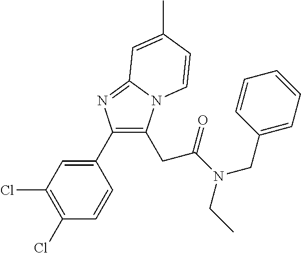 [SMILES: CCN(Cc1ccccc1)C(=O)Cc1c(-c2ccc(Cl)c(Cl)c2)nc2cc(C)ccn12] |
| 8 (YL- IPA08) | N-ethyl-N-(2-pyridylmethyl)-2-[2-(3,4- dichlorophenyl)-7-methyl-imidazo[1,2-a]pyridin-3- yl]-acetamide·hydrochloride | 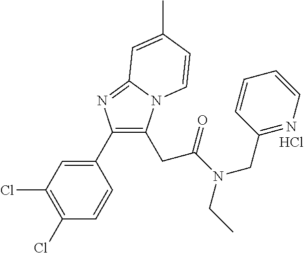 [SMILES: CCN(Cc1ccccn1)C(=O)Cc1c(-c2ccc(Cl)c(Cl)c2)nc2cc(C)ccn12 ; Cl] |
| 9 | N-ethyl-N-(3-pyridylmethyl)-2-[2-(3,4- dichlorophenyl)-7-methyl-imidazo[1,2-a]pyridin-3- yl]-acetamide•hydrochloride | 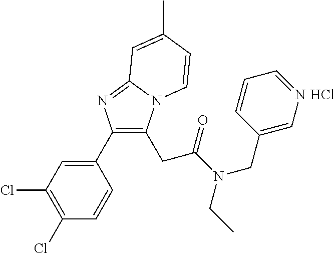 [SMILES: CCN(Cc1cccnc1)C(=O)Cc1c(-c2ccc(Cl)c(Cl)c2)nc2cc(C)ccn12 ; Cl] |
| 10 | N-ethyl-N-(4-pyridylmethyl)-2-[2-(3,4- dichlorophenyl)-7-methyl-imidazo[1,2-a]pyridin-3- yl]-acetamide•hydrochloride | 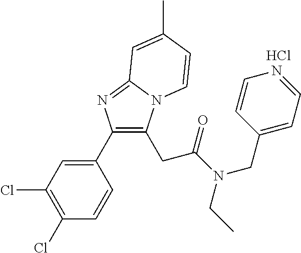 [SMILES: CCN(Cc1ccncc1)C(=O)Cc1c(-c2ccc(Cl)c(Cl)c2)nc2cc(C)ccn12 ; Cl] |
| 11 | N-(2-methoxyethyl)-N-(2-pyridylmethyl)-2-[2- (3,4-dichlorophenyl)-7-methyl-imidazo[1,2- a]pyridin-3-yl]-acetamide•hydrochloride | 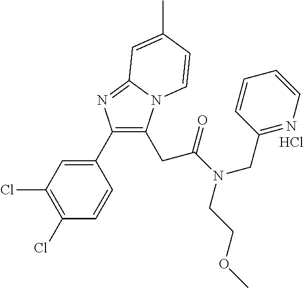 [SMILES: COCCN(Cc1ccccn1)C(=O)Cc1c(-c2ccc(Cl)c(Cl)c2)nc2cc(C)ccn12 ; Cl] |
| 12 | N-(2-methoxyethyl)-N-(3-pyridylmethyl)-2-[2- (3,4-dichlorophenyl)-7-methyl-imidazo[1,2- a]pyridin-3-yl]-acetamide·hydrochloride | 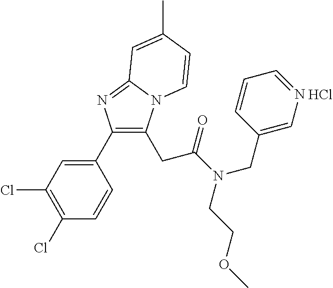 [SMILES: COCCN(Cc1cccnc1)C(=O)Cc1c(-c2ccc(Cl)c(Cl)c2)nc2cc(C)ccn12 ; Cl] |
| 13 | N-benzyl-N-ethyl-2-[2-(4-methylphenyl)-7-methyl- imidazo[1,2-a]pyridin-3-yl]-acetamide | 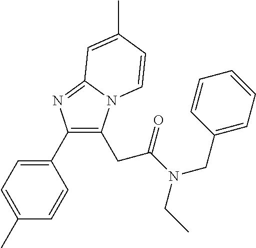 [SMILES: CCN(Cc1ccccc1)C(=O)Cc1c(-c2ccc(C)cc2)nc2cc(C)ccn12] |
| 14 | N-ethyl-N-(2-pyridylmethyl)-2-[2-(4- methylphenyl)-7-methyl-imidazo[1,2-a]pyridin-3- yl]-acetamide·hydrochloride | 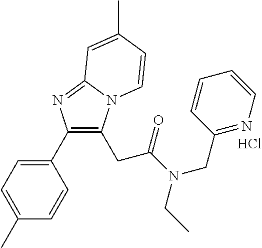 [SMILES: CCN(Cc1ccccn1)C(=O)Cc1c(-c2ccc(C)cc2)nc2cc(C)ccn12 ; Cl] |
| 15 | N-ethyl-N-(3-pyridylmethyl)-2-[2-(4- methylphenyl)-7-methyl-imidazo[1,2-a]pyridin-3- yl]-acetamide•hydrochloride | 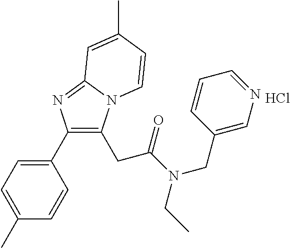 [SMILES: CCN(Cc1cccnc1)C(=O)Cc1c(-c2ccc(C)cc2)nc2cc(C)ccn12 ; Cl] |
| 16 | N-ethyl-N-(4-pyridylmethyl)-2-[2-(4- methylphenyl)-7-methyl-imidazo[1,2-a]pyridin-3- yl]-acetamide•hydrochloride | 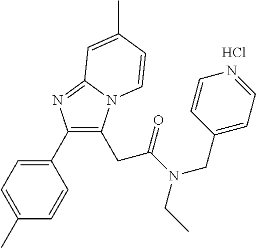 [SMILES: CCN(Cc1ccncc1)C(=O)Cc1c(-c2ccc(C)cc2)nc2cc(C)ccn12 ; Cl] |
| 17 | N-(2-methoxyethyl)-N-(2-pyridylmethyl)-2-[2-(4- methylphenyl)-7-methyl-imidazo[1,2-a]pyridin-3- yl]-acetamide·hydrochloride | 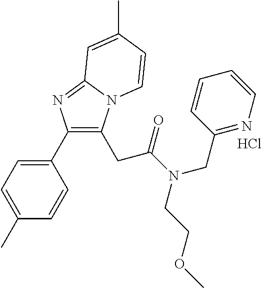 [SMILES: COCCN(Cc1ccccn1)C(=O)Cc1c(-c2ccc(C)cc2)nc2cc(C)ccn12 ; Cl] |
| 18 | N-(2-methoxyethyl)-N-(3-pyridylmethyl)-2-[2-(4- methylphenyl)-7-methyl-imidazo[1,2-a]pyridin-3- yl]-acetamide·hydrochloride | 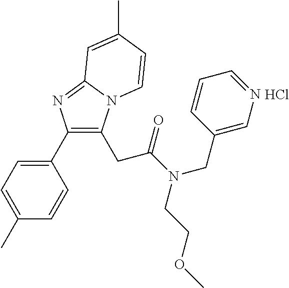 [SMILES: COCCN(Cc1cccnc1)C(=O)Cc1c(-c2ccc(C)cc2)nc2cc(C)ccn12 ; Cl] |
| 19 | N-benzyl-N-ethyl-2-[2-(4-methoxyphenyl)-7- methyl-imidazo[1,2-a]pyridin-3-yl]-acetamide | 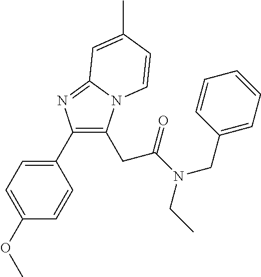 [SMILES: CCN(Cc1ccccc1)C(=O)Cc1c(-c2ccc(OC)cc2)nc2cc(C)ccn12] |
| 20 | N-ethyl-N-(2-pyridylmethyl)-2-[2-(4- methoxyphenyl)-7-methyl-imidazo[1,2-a]pyridin-3- yl]-acetamide hydrochloride | 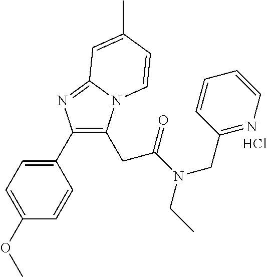 [SMILES: CCN(Cc1ccccn1)C(=O)Cc1c(-c2ccc(OC)cc2)nc2cc(C)ccn12 ; Cl] |
| 21 | N-ethyl-N-(3-pyridylmethyl)-2-[2-(4- methoxyphenyl)-7-methyl-imidazo[1,2-a]pyridin-3- yl]-acetamide·hydrochloride | 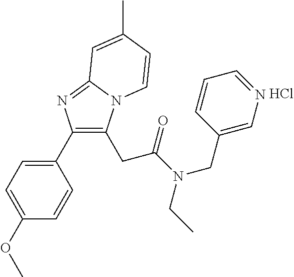 [SMILES: CCN(Cc1cccnc1)C(=O)Cc1c(-c2ccc(OC)cc2)nc2cc(C)ccn12 ; Cl] |
| 22 | N-ethyl-N-(4-pyridylmethyl)-2-[2-(4- methoxyphenyl)-7-methyl-imidazo[1,2-a]pyridin-3- yl]-acetamide•hydrochloride | 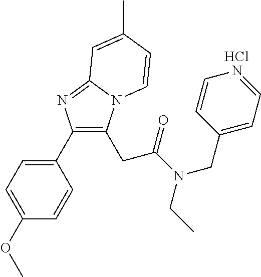 [SMILES: CCN(Cc1ccncc1)C(=O)Cc1c(-c2ccc(OC)cc2)nc2cc(C)ccn12 ; Cl] |
| 23 | N-(2-methoxyethyl)-N-(2-pyridylmethyl)-2-[2-(4- methoxyphenyl)-7-methyl-imidazo[1,2-a]pyridin-3- yl]-acetamide·hydrochloride | 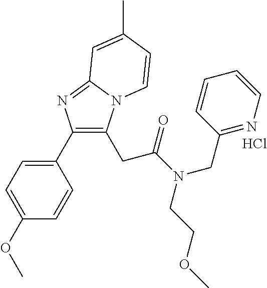 [SMILES: COCCN(Cc1ccccn1)C(=O)Cc1c(-c2ccc(OC)cc2)nc2cc(C)ccn12 ; Cl] |
| 24 | N-(2-methoxyethyl)-N-(3-pyridylmethyl)-2-[2-(4- methoxyphenyl)-7-methyl-imidazo[1,2-a]pyridin-3- yl]-acetamide·hydrochloride | 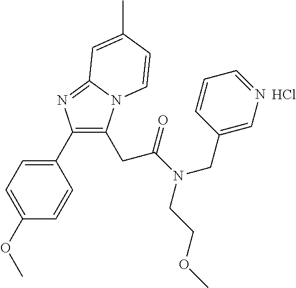 [SMILES: COCCN(Cc1cccnc1)C(=O)Cc1c(-c2ccc(OC)cc2)nc2cc(C)ccn12 ; Cl] |
| 25 | N-(2-methoxyethyl)-N-(2-morpholinoethyl)-2-[2- (4-methylphenyl)-7-methyl-imidazo[1,2-a]pyridin- 3-yl]-acetamide•hydrochloride | 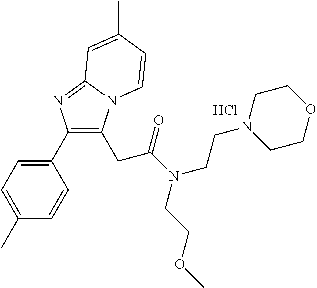 [SMILES: COCCN(CCN1CCOCC1)C(=O)Cc1c(-c2ccc(C)cc2)nc2cc(C)ccn12 ; Cl] |
| 26 | N-(2-methoxyethyl)-N-(2-morpholinoethyl)-2-[2- (4-methylphenyl)-6-methyl-imidazo[1,2-a]pyridin- 3-yl]-acetamide•hydrochloride | 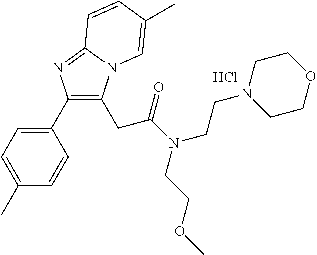 [SMILES: COCCN(CCN1CCOCC1)C(=O)Cc1c(-c2ccc(C)cc2)nc2ccc(C)cn12 ; Cl] |
| 27 | N-ethyl-N-(4-pyridylmethyl)-2-[2-(4- methylphenyl)-6-methyl-imidazo[1,2-a]pyridin-3- yl]-acetamide•hydrochloride | 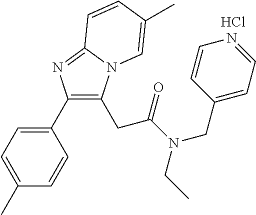 [SMILES: CCN(Cc1ccncc1)C(=O)Cc1c(-c2ccc(C)cc2)nc2ccc(C)cn12 ; Cl] |
| 28 | N-ethyl-N-(4-pyridylmethyl)-2-[2-(4- chlorophenyl)-6-methyl-imidazo[1,2-a]pyridin-3- yl]-acetamide•hydrochloride | 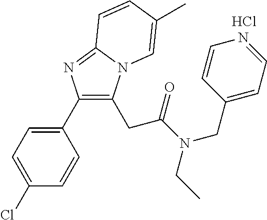 [SMILES: CCN(Cc1ccncc1)C(=O)Cc1c(-c2ccc(Cl)cc2)nc2ccc(C)cn12 ; Cl] |

Please refer to the Chinese Patent Invention Patent Publication No. CN102295642B for the preparation method of the above-mentioned compounds. The entire contents of this patent application are incorporated herein by reference.

In another aspect, the present application relates to use of a pharmaceutical composition comprising the compound of formula I, or the solvate or pharmaceutically acceptable salt thereof, as described in any preceding item, in the manufacture of a medicament for the prevention and/or treatment and/or adjuvant treatment of AD. The pharmaceutical composition may also comprise a pharmaceutically acceptable carrier or excipient.

The medicament is used for an animal, preferably a mammal, especially human. Typically, the pharmaceutical composition comprises 0.1-90% by weight of the compound of formula I and/or physiologically acceptable salt thereof. The pharmaceutical composition can be prepared according to the method known in the art. For this purpose, if desired, the compound of formula I, or the solvate or pharmaceutically acceptable salt thereof can be combined with one or more solid or liquid pharmaceutical excipients and/or adjuvants, and formulated to obtain an appropriate administration or dosage form for human use.

The compound of formula I, or the solvate or pharmaceutically acceptable salt thereof or the pharmaceutical composition comprising the same can be administered in unit dosage form, and the route of administration can be enteral or parenteral, such as oral, intramuscular, subcutaneous, nasal cavity, oral mucosa, skin, peritoneum or rectum, etc. Examples of dosage form include tablets, capsules, dropping pills, aerosols, pills, powders, solutions, suspensions, emulsions, granules, liposomes, transdermal agents, buccal tablets, suppositories, lyophilized powder injections, and the like, which can be general formulations, sustained-release formulations, controlled-release formulations and various microparticle delivery systems. In order to formulate the unit administration dosage form into tablets, various carriers well known in the art can be widely used. Examples of the carriers are, for example, diluents and absorbents, such as starch, dextrin, calcium sulfate, lactose, mannitol, sucrose, sodium chloride, glucose, urea, calcium carbonate, kaolin, microcrystalline cellulose, aluminum silicate, etc.; wetting agents and binders, such as water, glycerin, polyethylene glycol, ethanol, propanol, starch slurry, dextrin, syrup, honey, glucose solution, Arabic gum solution, gelatin solution, sodium carboxymethylcellulose, shellac, methylcellulose, potassium phosphate, polyvinylpyrrolidone, etc.; disintegrating agents, such as dry starch, alginate, agar powder, laminaran, sodium bicarbonate and citric acid, calcium carbonate, polyoxyethylene sorbitol fatty acid ester, dodecyl sodium sulfonate, methylcellulose, ethylcellulose, etc.; disintegration inhibitors, such as sucrose, glyceryl tristearate, cocoa butter, hydrogenated oil, etc.; absorption enhancers, such as quaternary ammonium salt, dodecyl sodium sulfate, etc.; lubricants, such as talc, silicon dioxide, corn starch, stearate, boric acid, liquid paraffin, polyethylene glycol, etc. The tablets can also be further prepared as coated tablets, such as sugar-coated, film-coated, enteric-coated, or bilayer and multi-layer tablets. In order to formulate the dosage unit into pills, various carriers well known in the art can be widely used. Examples of the carriers are, for example, diluents and absorbents, such as glucose, lactose, starch, cocoa butter, hydrogenated vegetable oil, polyvinylpyrrolidone, Gelucire, kaolin, talc, etc.; binders, such as Arabic gum, tragacanth, gelatin, ethanol, honey, liquid sugar, rice paste or batter, etc.; disintegrating agents, such as agar powder, dry starch, alginate, dodecyl sodium sulfonate, methylcellulose, ethylcellulose, etc. In order to formulate the dosage unit as a suppository, various carriers known in the art can be widely used. Examples of the carriers are, for example, polyethylene glycol, lecithin, cocoa butter, higher alcohols, esters of higher alcohols, gelatin, semi-synthetic glyceride and the like. In order to formulate the dosage unit into capsules, the active ingredient, i.e., the compound of formula I, or the solvate or pharmaceutically acceptable salt thereof, is mixed with the various carriers described above, and the resulting mixture is placed into hard gelatin capsules or soft capsules. The active ingredient, i.e., the compound of formula I, or the solvate or pharmaceutically acceptable salt, can also be prepared into microcapsules, suspended in an aqueous medium to form a suspension, and can also be filled into hard capsules or made into agents for injection. In order to formulate the dosage unit into injection preparations, such as solutions, emulsions, lyophilized powders and suspensions, all diluents commonly used in the art, for example, water, ethanol, polyethylene glycol, 1,3-propylene glycol, ethoxylated isostearyl alcohol, polyoxygenated isostearyl alcohol, polyoxyethylene sorbitan fatty acid ester, and the like can be used. In addition, in order to prepare an isotonic injection, an appropriate amount of sodium chloride, glucose or glycerol can be added to the injection preparation, and in addition, conventional cosolvent, buffer, pH adjuster and the like can be added.

In addition, if desired, colorants, preservatives, fragrances, flavors, sweeteners, or other materials can also be added to the pharmaceutical preparations.

The term “composition” as used herein is meant to include a product comprising specified ingredients each at specified amount, as well as any product that results, directly or indirectly, from combination of the specified ingredients each at specified amount.

In another aspect, the present application relates to a method for the prevention and/or treatment and/or adjuvant treatment of AD, comprising a step of administering a subject with an effective amount of the compound of formula I, or the solvate or pharmaceutically acceptable salt thereof as above-described or the pharmaceutical composition as above-described.

The dosage of the compound of formula I, or the solvate or pharmaceutically acceptable salt thereof, to be administered depends on many factors, such as the nature and severity of the disease to be prevented or treated, the sex, age, body weight and individual response of the patient or animal, the specific compound to be used, the route of administration and frequency of administration, etc. The above dosage may be administered in a single dosage form or divided into several, e.g., two, three or four dosage forms.

The actual dosage level of each active ingredient in the pharmaceutical composition can be varied so that the resulting amount of the active ingredient is effective for a particular patient, composition and route of administration to achieve the desired therapeutic response. The dosage level will be determined on the basis of the activity of the particular compound, the route of administration, the severity of the condition to be treated, and the condition and past medical history of the patient to be treated. However, the practice in the art is that the dose of the compound is starts from a level below the level required to obtain the desired therapeutic effect, and gradually increases the dose until the desired effect is obtained.

When used in the above-mentioned treatment and/or prevention or other treatments and/or prevention, a therapeutically and/or prophylactically effective amount of the compound may be used in pure form, or in the form of a pharmaceutically acceptable ester or prodrug (in the presence of such form). Alternatively, the compound may be administered in a pharmaceutical composition comprising the compound of interest together with one or more pharmaceutically acceptable excipients. The term “prophylactically and/or therapeutically effective amount” of the compound of the present invention refers to a sufficient amount of the compound to treat the disorder at a reasonable effect/risk ratio suitable for any medical prevention and/or treatment. It should be recognized that, however, the total daily dosage of the compound and the composition will be decided by the attending physician within the scope of sound medical judgment. For any particular patient, the particular therapeutically effective dosage level will depend upon a variety of factors, including the disorder being treated and the severity of the disorder; the activity of the particular compound to be used; the particular composition to be used; the age, weight, general health, sex, and diet of the patient; the time of administration, route of administration, and excretion rate of the particular compound; the duration of treatment; the drug used in combination or concomitantly with the particular compound; and similar factors well known in the medical field. For example, the practice in the art is that the dose of the compound is starts from a level below the level required to obtain the desired therapeutic effect, and gradually increases the dose until the desired effect is obtained. In general, the dosage of the compound of formula I for mammals, especially human, may be between 0.001-1000 mg/kg body weight/day, such as between 0.01-100 mg/kg body weight/day, such as between 0.01-10 mg/day kg body weight/day.

The term “subject” is preferably a mammal, more preferably human.

For all documents (including patent documents or non-patent documents) cited in the present invention, their entire contents are incorporated herein by reference, and if the meanings expressed in these documents are inconsistent with the present invention, the expressions of the present invention shall prevail. In addition, various terms and phrases used in the present invention have ordinary meanings known to those skilled in the art. Even so, the present invention still hopes to make more detailed descriptions and explanations for these terms and phrases. If the terms and phrases mentioned are inconsistent with the known meaning, the meaning expressed in the present invention shall prevail.

In the present invention, the term “halogen” refers to fluorine, chlorine, bromine or iodine.

In the present invention, the term “alkyl” has its ordinary meaning as known in the art, which is a straight or branched chain saturated hydrocarbyl. In a preferred embodiment of the present invention, the “C₁-C₆ alkyl group” refers to a saturated hydrocarbyl containing 1 to 6 (e.g., 1, 2, 3, 4, 5 or 6) carbon atoms, for example, but not limited to, methyl, ethyl, propyl, isopropyl, n-butyl, sec-butyl, tert-butyl, and the like.

In the present invention, the term “alkoxy” refers to a group having the structure of alkyl-O-, wherein alkyl is as defined above. In a preferred embodiment of the present invention, the “C₁-C₆ alkoxy” refers to a saturated hydrocarbonoxy group containing 1 to 6 (e.g., 1, 2, 3, 4, 5 or 6) carbon atoms, that is for example, but not limited to, methoxy, ethoxy, propoxy, isopropoxy, n-butoxy, sec-butoxy, tert-butoxy and the like.

The compound of Formula I described herein may exist in the form of a solvate, preferably a hydrate, which comprises a polar solvent, in particular water, methanol or ethanol, as a structural element of the compound of Formula I. The amount of the polar solvent, especially water, may be present in stoichiometric or non-stoichiometric ratio. It should be understood that, although solvates of the compound of formula I used in the treatment of AD may potentially provide different properties (including pharmacokinetic properties), upon the absorption into a subject, they will yield the compound of formula I, such that application of the compound of formula I respectively covers the application of any solvate of the compound of Formula I.

In the present invention, the term “TSPO (Translocator Protein)” refers to an 18 KD translocator protein, which is located in the outer mitochondrial membrane of microglia in the central nervous system, mediates cholesterol transport and synthesis of various neurosteroids, and is involved in the mitochondrial regulation, neuroinflammation, etc.

### Beneficial Effects of the Present Invention

TSPO ligands such as (2-aryl-imidazo[1,2-α]pyridin-3-yl)-acetamide derivatives, especially the compound YL-IPA08, are effective in the prevention and/or treatment and/or adjuvant treatment of AD, and can be used in the manufacture of a medicament for the prevention and/or treatment and/or adjuvant treatment of AD.

### BRIEF DESCRIPTION OF THE DRAWINGS

The accompanying drawings described herein are used to provide a further understanding of the present invention and constitute a part of the present application. The exemplary examples of the present invention and their descriptions are used to explain the present invention and do not constitute an improper limitation of the present invention. In the drawings:

FIG. 1 shows the experimental flow of Example 1.

FIG. 2 shows the water maze training results, i.e., the latency (a) and latency distance (b) for mice to find the platform. The results are expressed by x̅±SEM, *P<0.05, **P<0.01, ***P<0.001, each group was compared with the solvent control group of 5×FAD transgenic mice, and two-way ANOVA was used to detect the significant differences.

FIG. 3 shows the results of the water maze test, i.e., the number of times the mice crossed the platform (a) and the typical trajectories of the mice in each group in the maze (b). The results are expressed by x̅±SEM, *P<0.05, **P<0.01, and one-way ANOVA was used to detect the significant differences.

### SPECIFIC MODELS FOR CARRYING OUT THE INVENTION

The technical solutions in the examples of the present invention will be clearly and completely described below with reference to the accompanying drawings in the examples of the present invention. Obviously, the described examples are only a part of the examples of the present invention, but not all of the examples. The following description for at least one exemplary example is merely illustrative in nature and is in no way intended to limit the present invention, its application, or uses in any way. Based on the examples of the present invention, all other examples obtained by those of ordinary skill in the art without creative efforts shall fall within the scope of the present invention.

### Example 1

Animals: 5×FAD transgenic mice, male, SPF grade, initial body weight of 25-35 g; provided by Zhang Cheng Laboratory of Peking University. Male 5×FAD transgenic mice produced by breeding 5×FAD transgenic mice with WT mice and their littermate WT mice were used for the experiments, weighing 27-35 g. The 5×FAD transgenic mice were used as acute AD mouse model, the mice could develop AD pathological features at two or three months old, and behavioral changes appeared at five months old. The mice were administered at four months old, and after continuous administration for two months, the spatial learning and memory abilities of the mice were tested by water maze.

Test sample: YL-IPA08 (synthesized by the Institute of Toxic Drugs, Military Medical Research Institute, purity ≥99.8%).

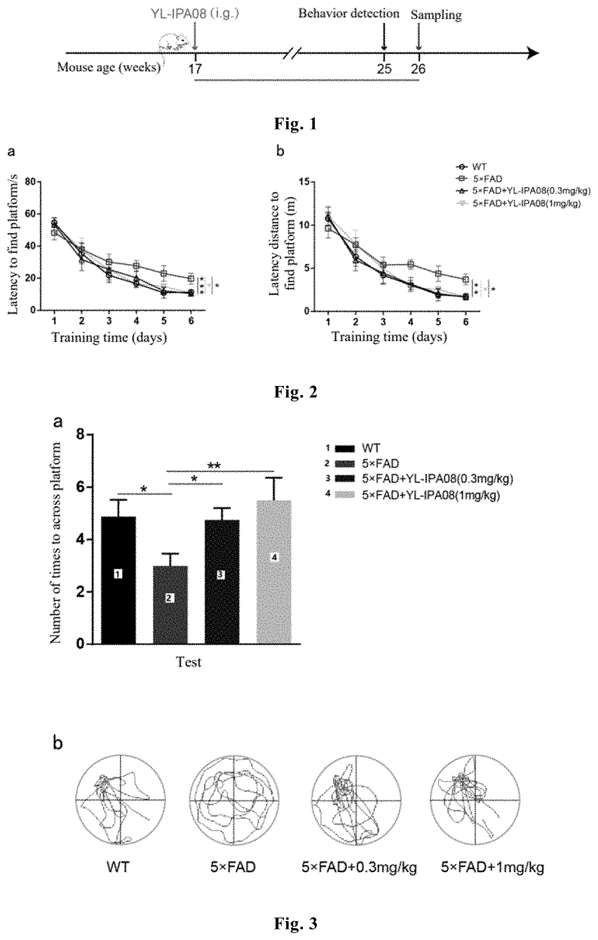

Methods (referring to FIG. 1):

(1) Administration: 4-month-old mice were divided into four groups, namely WT (physiological saline), 5×FAD (physiological saline), 5×FAD (YL-IPA08, 0.3 mg/kg), 5×FAD (YL-IPA08, 1 mg/kg), intragastrically (i.g.) administered, once a day. After continuous administration for 8 weeks, the spatial learning and memory ability of the mice were tested by Morris water maze.

(2) Morris water maze test: The Morris water maze test was performed after 8 weeks of the administration of YL-IPA08 (0.3, 1 mg/kg). The water maze consisted of a circular stainless steel pool with a diameter of 120 cm and a depth of 60 cm, and the diameter of the escape platform was 10 cm. The day before the test, the mice were allowed to swim in the water maze for 30 s-60 s to acclimate to the environment. During the training, the mice were allowed to swim freely for 60 s to find the platform. If the mice found the hidden platform autonomously, they were allowed to stay there for 30 seconds, and then taken out. If the mice could not find the platform autonomously, they were guided onto the platform and allowed to stay there for 30 seconds, and then taken out. Training was performed twice a day for 5-7 days until the latency of WT mice to find the platform no longer decreased, and the latency, distance and path of the mice to find the hidden platform in the water maze were recorded. The test was performed 24 hours after the last training session, in which the platform was removed, and the mice were allowed to explore freely in the water maze for 1 minute, and the number of times the mice crossed the position of the platform and the time they stayed in the quadrant of the platform were recorded. There were 8-10 mice per group.

Statistics and processing of data:

All data were presented by x̅±SEM. Data statistics and analysis were performed using GraphPad Prism 6.0. Significant differences were assessed by unpaired Student's t test, one-way ANOVA or two-way ANOVA followed by Tukey's multiple comparison test.

The results are shown in FIGS. 2 and 3.

In the Morris water maze test, we trained mice for 6 days, and found that during the training period, the distance and latency to find the platform were significantly longer in the 5×FAD transgenic mice group than in the WT group, while after being administrated with YL-IPA08, the distance and latency to find the platform in the 5×FAD transgenic mice were significantly shortened (FIG. 2). During the test period, we found that the number of times the mice in the administration group crossed the platform was significantly higher than that in the solvent control group, and the average swimming speed of the mice in each group was not significantly different (FIG. 3a). FIG. 3b shows the graph of representative trajectories of mice in each group during the test period. These results suggest that the TSPO agonist YL-IPA08 can improve learning and cognitive impairment in 5×FAD mice.

Various modifications of the present invention, in addition to those described herein, will be apparent to those skilled in the art from the foregoing description. Such modifications are also intended to fall within the scope of the appended claims. The references cited in the present application, including all patents, patent applications, journal articles, books, and any other publications, are incorporated by reference in its entirety.

What is claimed is:

1. Use of a compound of formula I, a solvate or pharmaceutically acceptable salt thereof, in the manufacture of a medicament for the prevention and/or treatment and/or adjuvant treatment of AD, 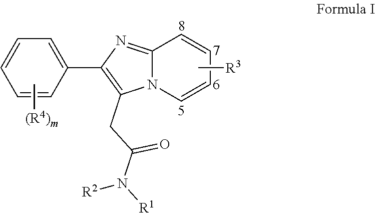
wherein:
R¹ is selected from the group consisting of ethyl, propyl, butyl and methoxyethyl;
R² is selected from the group consisting of benzyl, 2-pyridylmethyl, 3-pyridylmethyl, 4-pyridylmethyl, 2-(4-morpholinyl)ethyl and 3-(4-morpholinyl)propyl;
R³ is selected from the group consisting of H and C₁₋₆ alkyl;
each R⁴ is independently selected from the group consisting of H, halogen, C₁₋₆ alkyl and C₁-C₆ alkoxy;
m is selected from 0, 1, 2 or 3.

2. The use according to claim 1, wherein, R³ is selected from C₁₋₄ alkyl;
preferably, R³ is selected from methyl or ethyl;
preferably, R³ is on the 5-, 6-, 7- or 8-position of the imidazo[1,2-a]pyridine ring.

3. The use according to claim 1 or 2, wherein, R⁴ is selected from the group consisting of fluorine, chlorine, C₁₋₄ alkyl and C₁-C₄ alkoxy;
preferably, R⁴ is selected from the group consisting of fluorine, chlorine, methyl and methoxy, and m is 1 or 2;
preferably, R⁴ is chlorine, and m is 2.

4. The use according to any one of claims 1 to 3, wherein the pharmaceutically acceptable salt of the compound of formula I is the hydrochloride salt of the compound of formula I.

5. The use according to any one of claims 1 to 4, wherein the compound, or the solvate or pharmaceutically acceptable salt thereof is selected from:
N-benzyl-N-ethyl-2-[2-(4-chlorophenyl)-7-methyl-imidazo[1,2-a]pyridin-3-yl]-acetamide;
N-ethyl-N-(2-pyridylmethyl)-2-[2-(4-chlorophenyl)-7-methyl-imidazo[1,2-a]pyridin-3-yl]-acetamide⋅hydrochloride;
N-ethyl-N-(3-pyridylmethyl)-2-[2-(4-chlorophenyl)-7-methyl-imidazo[1,2-a]pyridin-3-yl]-acetamide⋅hydrochloride;
N-ethyl-N-(4-pyridylmethyl)-2-[2-(4-chlorophenyl)-7-methyl-imidazo[1,2-a]pyridin-3-yl]-acetamide⋅hydrochloride;
N-(2-methoxyethyl)-N-(2-pyridylmethyl)-2-[2-(4-chlorophenyl)-7-methyl-imidazo[1,2-a]pyridin-3-yl]-acetamide⋅hydrochloride;
N-(2-methoxyethyl)-N-(3-pyridylmethyl)-2-[2-(4-methylphenyl)-7-methyl-imidazo[1,2-a]pyridin-3-yl]-acetamide⋅hydrochloride;
N-benzyl-N-ethyl-2-[2-(3,4-dichlorophenyl)-7-methyl-imidazo[1,2-a]pyridin-3-yl]-acetamide;
N-ethyl-N-(2-pyridylmethyl)-2-[2-(3,4-dichlorophenyl)-7-methyl-imidazo[1,2-a]pyridin-3-yl]-acetamide⋅hydrochloride;
N-ethyl-N-(3-pyridylmethyl)-2-[2-(3,4-dichlorophenyl)-7-methyl-imidazo[1,2-a]pyridin-3-yl]-acetamide⋅hydrochloride;
N-ethyl-N-(4-pyridylmethyl)-2-[2-(3,4-dichlorophenyl)-7-methyl-imidazo[1,2-a]pyridin-3-yl]-acetamide⋅hydrochloride;
N-(2-methoxyethyl)-N-(2-pyridylmethyl)-2-[2-(3,4-dichlorophenyl)-7-methyl-imidazo[1,2-a]pyridin-3-yl]-acetamide⋅hydrochloride;
N-(2-methoxyethyl)-N-(3-pyridylmethyl)-2-[2-(3,4-dichlorophenyl)-7-methyl-imidazo[1,2-a]pyridin-3-yl]-acetamide⋅hydrochloride;
N-benzyl-N-ethyl-2-[2-(4-methylphenyl)-7-methyl-imidazo[1,2-a]pyridin-3-yl]-acetamide;
N-ethyl-N-(2-pyridylmethyl)-2-[2-(4-methylphenyl)-7-methyl-imidazo[1,2-a]pyridin-3-yl]-acetamide⋅hydrochloride;
N-ethyl-N-(3-pyridylmethyl)-2-[2-(4-methylphenyl)-7-methyl-imidazo[1,2-a]pyridin-3-yl]-acetamide⋅hydrochloride;
N-ethyl-N-(4-pyridylmethyl)-2-[2-(4-methylphenyl)-7-methyl-imidazo[1,2-a]pyridin-3-yl]-acetamide⋅hydrochloride;
N-(2-methoxyethyl)-N-(2-pyridylmethyl)-2-[2-(4-methylphenyl)-7-methyl-imidazo[1,2-a]pyridin-3-yl]-acetamide⋅hydrochloride;
N-(2-methoxyethyl)-N-(3-pyridylmethyl)-2-[2-(4-methylphenyl)-7-methyl-imidazo[1,2-a]pyridin-3-yl]-acetamide⋅hydrochloride;
N-benzyl-N-ethyl-2-[2-(4-methoxyphenyl)-7-methyl-imidazo[1,2-a]pyridin-3-yl]-acetamide;
N-ethyl-N-(2-pyridylmethyl)-2-[2-(4-methoxyphenyl)-7-methyl-imidazo[1,2-a]pyridin-3-yl]-acetamide⋅hydrochloride;
N-ethyl-N-(3-pyridylmethyl)-2-[2-(4-methoxyphenyl)-7-methyl-imidazo[1,2-a]pyridin-3-yl]-acetamide⋅hydrochloride;
N-ethyl-N-(4-pyridylmethyl)-2-[2-(4-methoxyphenyl)-7-methyl-imidazo[1,2-a]pyridin-3-yl]-acetamide⋅hydrochloride;
N-(2-methoxyethyl)-N-(2-pyridylmethyl)-2-[2-(4-methoxyphenyl)-7-methyl-imidazo[1,2-a]pyridin-3-yl]-acetamide⋅hydrochloride;
N-(2-methoxyethyl)-N-(3-pyridylmethyl)-2-[2-(4-methoxyphenyl)-7-methyl-imidazo[1,2-a]pyridin-3-yl]-acetamide⋅hydrochloride;
N-(2-methoxyethyl)-N-(2-morpholinoethyl)-2-[2-(4-methylphenyl)-7-methyl-imidazo[1,2-a]pyridin-3-yl]-acetamide⋅hydrochloride;
N-(2-methoxyethyl)-N-(2-morpholinoethyl)-2-[2-(4-methylphenyl)-6-methyl-imidazo[1,2-a]pyridin-3-yl]-acetamide⋅hydrochloride;
N-ethyl-N-(4-pyridylmethyl)-2-[2-(4-methylphenyl)-6-methyl-imidazo[1,2-a]pyridin-3-yl]-acetamide⋅hydrochloride; and
N-ethyl-N-(4-pyridylmethyl)-2-[2-(4-chlorophenyl)-6-methyl-imidazo[1,2-a]pyridin-3-yl]-acetamide⋅hydrochloride.

6. Use of a pharmaceutical composition comprising the compound, or the solvate or pharmaceutically acceptable salt thereof as defined in any one of claims 1 to 5 in the manufacture of a medicament for the prevention and/or treatment and/or adjuvant treatment of AD.

7. The use according to claim 6, wherein, the pharmaceutical composition further comprises a pharmaceutically acceptable carrier or excipient.

8. A method for the prevention and/or treatment and/or adjuvant treatment of AD, comprising a step of administrating a subject with an effective amount of the compound of formula I, or the solvate or pharmaceutically acceptable salt thereof or the pharmaceutical composition comprising the compound of formula I, or the solvate or pharmaceutically acceptable salt thereof, wherein the compound of formula I is as defined in any one of claims 1 to 5.
